(19) United States
(12) Patent Application Publication
(10) Pub. No.: US 2019/0022190 A1
(43) Pub. Date: Jan. 24, 2019

(54) GENERATION OF HEMOGLOBIN-BASED OXYGEN CARRIERS USING ELASTIN LIKE POLYPEPTIDES

(71) Applicant: University of Southern California, Los Angeles, CA (US)

(72) Inventors: Jordan Despanie, Los Angeles, CA (US); John Andrew MacKay, Pasadena, CA (US); Hao Guo, Los Angeles, CA (US)

(21) Appl. No.: 16/038,051

(22) Filed: Jul. 17, 2018

Related U.S. Application Data
(60) Provisional application No. 62/534,162, filed on Jul. 18, 2017.

(51) Int. Cl. A61K 38/42; C12N 15/62; G01N 33/72; A61P 7/08

(52) U.S. Cl. CPC A61K 38/42; A61P 7/08; G01N 33/721; C12N 15/62

(57) ABSTRACT

Provided herein is an agent comprising an elastin-like peptide (ELP) component fused to a hemoglobin protein, or a fragment of the hemoglobin protein, as well as methods of making an using same. The agents are useful as blood substitutes and treatment of anemia and related disorders.

### CROSS-REFERENCE TO RELATED APPLICATION

This application claims the benefit under 35 U.S.C. § 119(e) of U.S. Provisional Application No. 62/534,162, filed Jul. 18, 2017, the contents of which are incorporated by reference.

### BACKGROUND

Over 4.5 million patients require blood transfusions throughout North America each year. Blood transfusions are a life-saving intervention in a number of clinical settings such as battlefield hemorrhaging, major surgical procedures, and anemia. In events involving acute trauma, occurring in a serious car accident for example, a victim may need almost 100 pints of transfused blood. Transfusion therapy has therefore been an integral part of military medicine. As a vital component of blood, red blood cells (RBCs) are the most transfused blood product in battlefield trauma care; more than 54,000 units of RBCs are transfused every year in military hospitals.

### SUMMARY OF THE DISCLOSURE

Emergency circumstances usually need large amounts of blood, where donated blood may not be sufficient to fill the needs due to its limitation including storage issue and type compatibility issue, as well as its limited resources. But free hemoglobin released into the vasculature can rapidly scavenge nitric oxide and thus lead to systemic vasoconstriction, decreased blood flow, increased release of pro-inflammatory mediators and potent vasoconstrictors and a loss of platelet inactivation. Renal toxicity can also be induced by rapid infiltration of free hemoglobin from the kidney due to its low molecular weight. To address this problem, Applicant fused hemoglobin to one particle elastin-like polypeptide to prevent extravasation and reduce the nitric oxide scavenging-mediated toxicities.

Thus, provided herein is an agent comprising, or consisting essentially of, or consisting of an elastin-like peptide (ELP) component fused to a hemoglobin protein, or a fragment of the hemoglobin protein. In one aspect, the agent further comprises a detectable label. In another aspect, the agent further comprises a purification label. The hemoglobin protein and fragment thereof can be from any suitable species, e.g., an animal, a mammalian, a canine, a feline, a murine, a bovine, an equine, or a human patient.

In one aspect, the hemoglobin fragment is an alpha or beta subunit of the hemoglobin protein. In another aspect, the hemoglobin protein comprises, consists essentially of, or yet further consists of the alpha and beta subunits. Exemplary amino acid sequences for the hemoglobin protein and fragments thereof are provided in the sequence listing. Alternative exemplary sequences are known in the art, e.g. UniProtKB—P69905 (HBA_Human), (uniprot.org/uniprot/P69905, last accessed on Jul. 9, 2016); UniProtKB—P68871 (HBB_Human), (uniprot.org/uniprot/P68871, last accessed on Jul. 9, 2016); HGNC:4827 (beta subunit, genenames.org/cgi-bin/gene_symbol_report?hgnc_id=HGNC:4827, last accessed on Jul. 9, 2016, and homologs disclosed therein).

In another aspect, the ELP of the agent comprises, or consists essentially of, or yet further consists of a reference polypeptide (VPGXG)n, (wherein n is an integer that denotes the number of repeats, and can be from about between 5 and 400, alternatively between 5 and 300, or alternatively between 25 and 250, or alternatively between 25 and 150, or from about 6 to about 200, or alternatively from about 15 to 195, or alternatively from 40 to about 195, or alternatively about 24, or alternatively about 48, or alternatively about 96, or alternatively about 192, and X is an amino acid selected from Ser, Ala, Ile, or Val, or a biological equivalent thereof, wherein a biological equivalent of the reference polypeptide is a peptide that has at least 80% sequence identity to the reference polypeptide or a peptide encoded by a polynucleotide that hybridizes under conditions of high stringency to a polynucleotide that encodes the reference polypeptide or its complement, wherein conditions of high stringency comprise hybridization reaction at about 60° C. in about 1×SSC.

In a yet further aspect, the ELP of the agent comprises, or consists essentially of, or yet further consist of, a reference polypeptide designated A192 or a biological equivalent thereof, wherein a biological equivalent of the reference polypeptide is a peptide that has at least 80% sequence identity to the reference polypeptide or a peptide encoded by a polynucleotide that hybridizes under conditions of high stringency to a polynucleotide that encodes the reference polypeptide or its complement, wherein conditions of high stringency comprise hybridization reaction at about 60° C. in about 1×SSC.

Further provided are isolated polynucleotides encoding the agents as described herein. The polynucleotides can further comprise a detectable label and can be contained within a vector, such as a viral vector or plasmid. They can further comprise, or alternatively consist essentially of, or yet further consist of, regulatory agents, such as promoters or enhancers that assist with expression of the polynucleotides. In a further aspect, the vector is the plasmid is hemoglobin_on pet25.

In a further aspect, provided herein is a host cell comprising the isolated polynucleotide and/or vector as described herein. The host cell can be a eukaryotic cell or a prokaryotic cell, e.g., an E. coli cell.

In another aspect, provided herein is a method for preparing an agent, comprising, or alternatively consisting essentially of, or yet further consisting of, growing or culturing the host cell comprising a polynucleotide as described herein, under conditions that favor expression of a polynucleotide to the agent. In a further aspect, the method further comprises, or consists essentially of, or yet further consists of isolating the agent.

Compositions are further provided herein In one aspect, the composition comprises, or consists essentially of, or yet further consists of the agent as described herein and a carrier. In another aspect, the composition comprises, or consists essentially of, or yet further consists of the isolated polynucleotide, vector and/or host cell as described herein and a carrier. A non-limiting example of a carrier is a pharmaceutically acceptable carrier. The compositions and agents are useful as a blood substitute.

The agents and compositions as described herein are useful to treat a patient or subject in need thereof, comprising, or alternatively consisting essentially of, or yet further consisting of, administering an effective amount of the agent as described herein, thereby treating the patient or subject. In one aspect, the agent is administered intravenously. The patient or subject is a mammal, e.g., a human, a canine, a feline, an equine, and the hemoglobin of the agent is of the same species of as the patient or subject being treated, e.g, a human hemoglobin is used to treat a human patient.

The patient or subject in need of the agent is suffering from a blood disorder or may be in the need of a transfusion. Thus, the method can be used to treat anemia, hemophilia, thalassemia, sickle cell anemia, or malaria by administering an effective amount of the agent as described herein.

Kits and screens using the agents or compositions as described herein, are further provided herein.

### BRIEF DESCRIPTION OF THE DRAWINGS

FIG. 1: The scheme of chemical conjugation approach. The addition of Sulfo-NHS can increase the reaction efficiency.

FIG. 2: The SDS-PAGE demonstrating the shift of molecular weight after chemical conjugation. In the first two lanes loaded by fusion protein, two bands can be seen above the bands of plain A192, which are believed to be A192 binding to one or two subunit of hemoglobin.

FIG. 3: The result of SEC purification. Two peaks can be observed, which represents fusion and plain A192 respectively.

FIG. 4: The DLS results revealing the different hydrodynamic radii of two different SEC fractions. In fraction 1, which we think is fusion protein, nanoparticles with a hydrodynamic radius of 11.4 nm represent 99.7% of total mass. In fraction 2, which represents plain A192, nanoparticles are monodispersed and have a hydrodynamic radius of 7.4 nm.

FIG. 5: The result of UV-Vis temperature ramp showing that the phase separation property of ELPs is well maintained after conjugation.

FIG. 6: Map of Hb_on Pet 25.

### BRIEF DESCRIPTION OF THE SEQUENCE LISTING

Attached to this disclosure, preceding the claims are exemplary sequences that are referenced herein.

### DETAILED DESCRIPTION

### Definitions

The practice of the present disclosure will employ, unless otherwise indicated, conventional techniques of tissue culture, immunology, molecular biology, microbiology, cell biology and recombinant DNA, which are within the skill of the art. See, e.g., Sambrook et al., (1989) Molecular Cloning: A Laboratory Manual, 2nd edition; Ausubel et al., eds. (1987) Current Protocols In Molecular Biology; MacPherson, B. D. Hames and G. R. Taylor eds., (1995) PCR 2: A Practical Approach; Harlow and Lane, eds. (1988) Antibodies, A Laboratory Manual; Harlow and Lane, eds. (1999) Using Antibodies, a Laboratory Manual; and R. I. Freshney, ed. (1987) Animal Cell Culture.

All numerical designations, e.g., pH, temperature, time, concentration, and molecular weight, including ranges, are approximations which are varied (+) or (−) by increments of 1.0 or 0.1, as appropriate. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a,” “an” and “the” include plural references unless the context clearly dictates otherwise.

As used herein, the term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of” shall mean excluding more than trace elements of other ingredients and substantial method steps. Embodiments defined by each of these transition terms are within the scope of this disclosure.

A “composition” is also intended to encompass a combination of active agent and another carrier, e.g., compound or composition, inert (for example, a detectable agent or label) or active, such as an adjuvant, diluent, binder, stabilizer, buffers, salts, lipophilic solvents, preservative, adjuvant or the like. In the context of this application, the active agent is the ELP-containing a ligand and therapeutic agent as described herein. Carriers also include pharmaceutical excipients and additives proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this disclosure, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A “pharmaceutical composition” is intended to include the combination of an active agent with a carrier, inert or active, making the composition suitable for diagnostic or therapeutic use in vitro, in vivo or ex vivo.

The term “pharmaceutically acceptable carrier” (or medium), which may be used interchangeably with the term biologically compatible carrier or medium, refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio. Pharmaceutically acceptable carriers suitable for use in the present disclosure include liquids, semi-solid (e.g., gels) and solid materials (e.g., cell scaffolds and matrices, tubes sheets and other such materials as known in the art and described in greater detail herein). These semi-solid and solid materials may be designed to resist degradation within the body (non-biodegradable) or they may be designed to degrade within the body (biodegradable, bioerodable). A biodegradable material may further be bioresorbable or bioabsorbable, i.e., it may be dissolved and absorbed into bodily fluids (water-soluble implants are one example), or degraded and ultimately eliminated from the body, either by conversion into other materials or breakdown and elimination through natural pathways.

As used herein, the term “patient” or “subject” intends an animal, a mammal or yet further a human patient. For the purpose of illustration only, a mammal includes but is not limited to a human, a feline, a canine, a simian, a murine, a bovine, an equine, a porcine or an ovine.

The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

The term “therapeutic” refers to an agent or component capable of inducing a biological effect in vivo and/or in vitro. The biological effect may be useful for treating and/or preventing a condition, disorder, or disease in a subject or patient. A therapeutic may include, without limitation, a small molecule, a nucleic acid, or a polypeptide. Non-limiting examples of such include rapamycin and cyclosporin A. In one aspect, “therapeutic” excludes preventive use.

As used herein, the term “elastin-like peptide (ELP) component” intends a polypeptide that forms stable nanoparticle (also known as a micelle) above the transition temperature of the ELP. In one aspect, the ELP component comprises, or alternatively consists essentially of, or yet further consists of the polypeptide (VPGXG)n, wherein X is any amino acid, or alternatively Ala, Ser, Ile or Val, and wherein n is an integer that denotes the number of repeats, and can be from about between 5 and 400, alternatively between 5 and 300, or alternatively between 25 and 250, or alternatively between 25 and 150, or from about 6 to about 200, or alternatively from about 15 to 195, or alternatively from 40 to about 195, or alternatively about 24, or alternatively about 48, or alternatively about 96, or alternatively about 192. In one aspect, the ELP is A192: G(VPGAG)₁₉₂Y or A96: G(VPGAG)₉₆Y, or an equivalent of each thereof. In another aspect the ELP has the sequence G(VPGAG)n(VPGIG)nY, (wherein n is an integer that denotes the number of repeats, and can be from about between 5 and 400, alternatively between 5 and 300, or alternatively between 25 and 250, or alternatively between 25 and 150, or from about 6 to about 200, or alternatively from about 15 to 195, or alternatively from 40 to about 195, or alternatively about 24, or alternatively about 48, or alternatively about 96, or alternatively about 192, wherein in one aspect, the ELP comprises, or alternatively consists essentially of, or yet further consists of the amino acid sequence A96I96: G(VPGAG)₉₆(VPGIG)₉₆Y, or a biological equivalent thereof. A biological equivalent of an ELP polypeptide is a peptide that has at least 80% sequence identity to the reference polypeptide or a peptide encoded by a polynucleotide that hybridizes under conditions of high stringency to a polynucleotide that encodes ELP polypeptide or its complement, wherein conditions of high stringency comprise hybridization reaction at about 60° C. in about 1×SSC. In one aspect, the biological equivalent will retain the characteristic or function of forming a nanoparticle (also known as a micelle) when the biological equivalent is raised above the transition temperature of the biological equivalent or, for example, the transition temperature of an ELP as described herein.

As used herein, the term “biological equivalent thereof” is used synonymously with “equivalent” unless otherwise specifically intended. When referring to a reference protein, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 60%, or 65%, or 70%, or 75%, or 80% homology or identity and alternatively, at least about 85%, or alternatively at least about 90%, or alternatively at least about 95%, or alternatively 98% percent homology or identity and exhibits substantially equivalent biological activity to the reference protein, polypeptide or nucleic acid. Alternatively, a biological equivalent is a peptide encoded by a nucleic acid that hybridizes under stringent conditions to a nucleic acid or complement that encodes the peptide or with respect to polynucleotides, those hybridize under stringent conditions to the reference polynucleotide or its complement. Hybridization reactions can be performed under conditions of different “stringency”. In general, a low stringency hybridization reaction is carried out at about 40° C. in about 10×SSC or a solution of equivalent ionic strength/temperature. A moderate stringency hybridization is typically performed at about 50° C. in about 6×SSC, and a high stringency hybridization reaction is generally performed at about 60° C. in about 1×SSC. Hybridization reactions can also be performed under “physiological conditions” which is well known to one of skill in the art. A non-limiting example of a physiological condition is the temperature, ionic strength, pH and concentration of Mg²⁺ normally found in a cell.

A polynucleotide or polynucleotide region (or a polypeptide or polypeptide region) having a certain percentage (for example, about 60%, 65%, 70%, 75%, 80%, 85%, 90%, 95% or 97%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

An “equivalent” of a polynucleotide or polypeptide refers to a polynucleotide or a polypeptide having a substantial homology or identity to the reference polynucleotide or polypeptide. In one aspect, a “substantial homology” is greater than about 60%, 65%, 70%, 75%, 80%, 85%, 90%, 95% or 98% homology.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

“Regulatory polynucleotide sequences” intends any one or more of promoters, operons, enhancers, as known to those skilled in the art to facilitate and enhance expression of polynucleotides.

An “expression vehicle” is a vehicle or a vector, non-limiting examples of which include viral vectors or plasmids, that assist with or facilitate expression of a gene or polynucleotide that has been inserted into the vehicle or vector.

A “delivery vehicle” is a vehicle or a vector that assists with the delivery of an exogenous polynucleotide into a target cell. The delivery vehicle may assist with expression or it may not, such as traditional calcium phosphate transfection compositions.

“An effective amount” refers to the amount of an active agent or a pharmaceutical composition sufficient to induce a desired biological and/or therapeutic result. That result can be alleviation of the signs, symptoms, or causes of a disease, or any other desired alteration of a biological system. The effective amount will vary depending upon the health condition or disease stage of the subject being treated, timing of administration, the manner of administration and the like, all of which can be determined readily by one of ordinary skill in the art.

As used herein, the terms “treating,” “treatment” and the like are used herein to mean obtaining a desired pharmacologic and/or physiologic effect. The effect may be prophylactic in terms of completely or partially preventing a disorder or sign or symptom thereof, and/or may be therapeutic in terms of a partial or complete cure for a disorder and/or adverse effect attributable to the disorder. In one aspect, the term excludes prevention or prophylaxis.

As used herein, to “treat” further includes systemic amelioration of the symptoms associated with the pathology and/or a delay in onset of symptoms. Clinical and sub-clinical evidence of “treatment” will vary with the pathology, the subject, and the treatment.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, injection, topical application, intraperitoneal, intravenous and by inhalation. An agent of the present disclosure can be administered for therapy by any suitable route of administration. It will also be appreciated that the preferred route will vary with the condition and age of the recipient, and the disease being treated.

The agents and compositions of the present disclosure can be used in the manufacture of medicaments and for the treatment of humans and other animals by administration in accordance with conventional procedures, such as an active ingredient in pharmaceutical compositions.

As used herein, the term “patient” or “subject” intends an animal, a mammal or yet further a human patient. For the purpose of illustration only, a mammal includes but is not limited to a human, a feline, a canine, a simian, a murine, a bovine, an equine, a porcine or an ovine. In terms of cells, the term “mammalian cells” includes, but is not limited to cells of the following origin: a human, a feline, a canine, a simian, a murine, a bovine, an equine, a porcine or an ovine.

As used herein, the term “detectable label” intends a directly or indirectly detectable compound or composition that is conjugated directly or indirectly to the composition to be detected, e.g., N-terminal histidine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition that is detectable. The labels can be suitable for small scale detection or more suitable for high-throughput screening. As such, suitable labels include, but are not limited to magnetically active isotopes, non-radioactive isotopes, radioisotopes, fluorochromes, luminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescent labeling assay components are known in the art and described for example in Haugland, Richard P. (1996) Handbook of Fluorescent Probes and Research Chemicals (6^{th} ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6^{th} ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

“Hemoglobin” is the iron-containing oxygen transport metalloprotein in red blood cells of all vertebrates and in some invertebrates. The protein is a tetramer composed of two types of subunits designated alpha and beta, with stoichiometry α2β2. The four subunits of hemoglobin sit roughly at the corners of a tetrahedron, facing each other across a cavity of the center of the molecule.

### Elastin-Like Polypeptides (ELPs)

Elastin-like-polypeptides (ELPs) are a genetically engineered polypeptide with unique phase behavior (see for e.g. S. R. MacEwan, et al., Biopolymers 94(1) (2010) 60-77), which promotes recombinant expression, protein purification, and self-assembly of nanostructures (see for e.g. A. Chilkoti, et al., Advanced Drug Delivery Reviews 54 (2002) 1093-1111). ELPs are artificial polypeptides composed of repeated pentapeptide sequences, (Val-Pro-Gly-Xaa-Gly)n derived from human tropoelastin, where Xaa is the “guest residue” which is any amino acid, an amino acid analog or amino acid derivative thereof. In one embodiment, Xaa is any amino acid except proline. In another aspect, the guest residue is Ile, Val, Ala or Ser This peptide motif displays rapid and reversible de-mixing from aqueous solutions above a transition temperature, Tₜ. Below Tₜ, ELPs adopt a highly water soluble random coil conformation; however, above Tₜ, they separate from solution, coalescing into a second aqueous phase. The Tₜ of ELPs can be tuned by choosing the guest residue and ELP chain length as well as fusion peptides at the design level (see for e.g. MacEwan S R, et al., Biopolymers 94(1): 60-77). The ELP phase is both biocompatible and highly specific for ELPs or ELP fusion proteins, even in complex biological mixtures. Genetically engineered ELPs are monodisperse, biodegradable, non-toxic. Throughout this description, ELPs are identified by the single letter amino acid code of the guest residue followed by the number of repeat units, n.

Described herein are ELP fusion proteins, which can be self-assembled into nanoparticles (alternatively known as micelles). The diameter of the nanoparticle can be from about 1 to about 1000 nm or from about 1 to about 500 nm, or from about 1 to about 100 nm, or from about 1 to about 50 nm, or from about 20 to about 50 nm, or from about 30 to about 50 nm, or from about 35 to about 45 nm, or from about 4 to about 30 nm, or from about 8 to about 30 nm, or about 9 to 25 nm. In one embodiment, the diameter is about 40 nm. The fusion proteins are composed of elastin-like-polypeptides and high affinity polypeptides. These fusion proteins can be expressed from a variety of expression systems known to those skilled in the art and easily purified by the phase transition behavior of ELPs.

ELPs have potential advantages over chemically synthesized polymers as drug delivery agents. First, because they are biosynthesized from a genetically encoded template, ELPs can be made with precise molecular weight. Chemical synthesis of long linear polymers does not typically produce an exact length, but instead a range of lengths. Consequently, fractions containing both small and large polymers yield mixed pharmacokinetics and bio-distribution. Second, ELP biosynthesis produces very complex amino acid sequences with nearly perfect reproducibility. This enables very precise selection of the location of drug attachment. Thus drug can be selectively placed on the corona, buried in the core, or dispersed equally throughout the polymer. Third, ELP can self-assemble into multivalent nanoparticles that can have excellent site-specific accumulation and drug carrying properties. Fourth, because ELP are designed from native amino acid sequences found extensively in the human body they are biodegradable, biocompatible, and tolerated by the immune system. Fifth, ELPs undergo an inverse phase transition temperature, Tₜ, above which they phase separate into large aggregates. By localized heating, additional ELP can be drawn into the target site, which may be beneficial for increasing drug concentrations.

In addition to therapeutics, the ELPs may also be associated with a detectable label that allows for the visual detection of in vivo uptake of the ELPs. Suitable labels include, for example, fluorescein, rhodamine, tetramethylrhodamine, eosin, erythrosin, coumarin, methyl-coumarins, pyrene, Malacite green, Alexa-Fluor®, stilbene, Lucifer Yellow, Cascade Blue™, and Texas Red. Other suitable optical dyes are described in Haugland, Richard P. (1996) Molecular Probes Handbook.

In certain embodiments, the ELP components include polymeric or oligomeric repeats of the pentapeptide (VPGXG)n, wherein n is an integer representing the number of repeats between 5 and 400, alternatively between 5 and 300, or alternatively between 25 and 250, or alternatively between 25 and 150, or from about 6 to about 200, or alternatively from about 15 to 195, or alternatively from 40 to about 195, or alternatively about 24, or alternatively about 48, or alternatively about 96, or alternatively about 192, and wherein the guest residue X (also denoted as Xaa herein) is any amino acid, that in one aspect, excludes proline. X may be a naturally occurring or non-naturally occurring amino acid. In some embodiments, X is selected from alanine, arginine, asparagine, aspartic acid, cysteine, glutamic acid, glutamine, glycine, histidine, isoleucine, leucine, lysine, methionine, phenylalanine, serine, threonine, tryptophan, tyrosine and valine. In some embodiments, X is a natural amino acid other than proline or cysteine. In one aspect, it is Ala, Val, Ser, or Ile.

The guest residue X may be a non-classical (non-genetically encoded) amino acid. Examples of non-classical amino acids include: D-isomers of the common amino acids, 2, 4-diaminobutyric acid, α-amino isobutyric acid, A-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, C α-methyl amino acids, N α-methyl amino acids, and amino acid analogs in general.

Selection of X is independent in each ELP structural unit (e.g., for each structural unit defined herein having a guest residue X). For example, X may be independently selected for each structural unit as an amino acid having a positively charged side chain, an amino acid having a negatively charged side chain, or an amino acid having a neutral side chain, including in some embodiments, a hydrophobic side chain.

In each embodiment, the structural units, or in some cases polymeric or oligomeric repeats, of the ELP sequences may be separated by one or more amino acid residues that do not eliminate the overall effect of the molecule, that is, in imparting certain improvements to the therapeutic component as described. In certain embodiments, such one or more amino acids also do not eliminate or substantially affect the phase transition properties of the ELP component (relative to the deletion of such one or more amino acids).

The ELP component in some embodiments is selected or designed to provide a Tₜ ranging from about 10 to about 80° C., such as from about 35 to about 60° C., or from about 38 to about 45° C. In some embodiments, the Tₜ is greater than about 40° C. or greater than about 42° C., or greater than about 45° C., or greater than about 50° C. The transition temperature, in some embodiments, is above the body temperature of the subject or patient (e.g., >37° C.) thereby remaining soluble in vivo, or in other embodiments, the Tₜ is below the body temperature (e.g., <37° C.) to provide alternative advantages, such as in vivo formation of a drug depot for sustained release of the therapeutic agent.

The Tₜ of the ELP component can be modified by varying ELP chain length, as the Tt generally increases with decreasing MW. For polypeptides having a molecular weight >100,000, the hydrophobicity scale developed by Urry et al. (PCT/US96/05186, which is hereby incorporated by reference in its entirety) is preferred for predicting the approximate Tₜ of a specific ELP sequence. However, in some embodiments, ELP component length can be kept relatively small, while maintaining a target Tₜ, by incorporating a larger fraction of hydrophobic guest residues (e.g., amino acid residues having hydrophobic side chains) in the ELP sequence. For polypeptides having a molecular weight <100,000, the Tₜ may be predicted or determined by the following quadratic function: Tₜ=M₀+M₁X+M₂X² where X is the MW of the fusion protein, and M₀=116.21; M₁=−1.7499; M₂=0.010349.

While the Tₜ of the ELP component, and therefore of the ELP component coupled to a therapeutic component, is affected by the identity and hydrophobicity of the guest residue, X, additional properties of the molecule may also be affected. Such properties include, but are not limited to solubility, bioavailability, persistence, and half-life of the molecule.

### Expression of Recombinant Proteins

ELPs and other recombinant proteins described herein can be prepared by expressing polynucleotides encoding the polypeptide sequences of this disclosure in an appropriate host cell, i.e., a prokaryotic or eukaryotic host cell This can be accomplished by methods of recombinant DNA technology known to those skilled in the art. It is known to those skilled in the art that modifications can be made to any peptide to provide it with altered properties. Polypeptides of the disclosure can be modified to include unnatural amino acids. Thus, the peptides may comprise D-amino acids, a combination of D- and L-amino acids, and various “designer” amino acids (e.g., β-methyl amino acids, C-α-methyl amino acids, and N-α-methyl amino acids, etc.) to convey special properties to peptides. Additionally, by assigning specific amino acids at specific coupling steps, peptides with α-helices, β turns, β sheets, α-turns, and cyclic peptides can be generated. Generally, it is believed that beta-turn spiral secondary structure or random secondary structure is preferred.

The ELPs can be expressed and purified from a suitable host cell system. Suitable host cells include prokaryotic and eukaryotic cells, which include, but are not limited to bacterial cells, yeast cells, insect cells, animal cells, mammalian cells, murine cells, rat cells, sheep cells, simian cells and human cells. Examples of bacterial cells include Escherichia coli, Salmonella enterica and Streptococcus gordonii. In one embodiment, the host cell is E. coli. The cells can be purchased from a commercial vendor such as the American Type Culture Collection (ATCC, Rockville Md., USA) or cultured from an isolate using methods known in the art.

Examples of suitable eukaryotic cells include, but are not limited to 293T HEK cells, as well as the hamster cell line BHK-21; the murine cell lines designated NIH3T3, NS0, C127, the simian cell lines COS, Vero; and the human cell lines HeLa, PER.C6 (commercially available from Crucell) U-937 and Hep G2. A non-limiting example of insect cells includes Spodoptera frugiperda. Examples of yeast useful for expression include, but are not limited to Saccharomyces, Schizosaccharomyces, Hansenula, Candida, Torulopsis, Yarrowia, or Pichia. See e.g., U.S. Pat. Nos. 4,812,405; 4,818,700; 4,929,555; 5,736,383; 5,955,349; 5,888,768 and 6,258,559.

### Protein Purification

The phase transition behavior of the ELPs allows for easy purification. The ELPs may also be purified from host cells using methods known to those skilled in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide or polypeptide are filtration, ion-exchange chromatography, exclusion chromatography, polyacrylamide gel electrophoresis, affinity chromatography, or isoelectric focusing. A particularly efficient method of purifying peptides is fast protein liquid chromatography or even HPLC. In the case of ELP compositions protein purification may also be aided by the thermal transition properties of the ELP domain as described in U.S. Pat. No. 6,852,834.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “[n]-fold purification number” wherein “n” is an integer. The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxyapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

### Pharmaceutical Compositions

Pharmaceutical compositions are further provided. The compositions comprise a carrier and an agent, an ELP with the hemoglobin protein or fragment thereof, or a polynucleotide encoding the ELP, as described herein or other compositions (e.g., polynucleotide, vector system, host cell) as described herein. The carriers can be one or more of a solid support or a pharmaceutically acceptable carrier. In one aspect, the compositions are formulated with one or more pharmaceutically acceptable excipients, diluents, carriers and/or adjuvants. In addition, embodiments of the compositions include ELPs, formulated with one or more pharmaceutically acceptable auxiliary substances.

The disclosure provides pharmaceutical formulations in which the one or more of an agent, ELP-fusion with a ligand, or a polynucleotide, vector or host cells can be formulated into preparations for injection or other appropriate route of administration in accordance with the disclosure by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives or other antimicrobial agents.

Aerosol formulations provided by the disclosure can be administered via inhalation. For example, embodiments of the pharmaceutical formulations of the disclosure comprise a compound of the disclosure formulated into pressurized acceptable propellants such as dichlorodifluoromethane, propane, nitrogen and the like.

Embodiments of the pharmaceutical formulations of the disclosure include those in which the composition is formulated in an injectable composition. Injectable pharmaceutical formulations of the disclosure are prepared as liquid solutions or suspensions; or as solid forms suitable for solution in, or suspension in, liquid vehicles prior to injection. The preparation may also be emulsified or the active ingredient encapsulated in liposome vehicles in accordance with other embodiments of the pharmaceutical formulations of the disclosure.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Methods of preparing such dosage forms are known, or will be apparent upon consideration of this disclosure, to those skilled in the art. See, e.g., Remington's Pharmaceutical Sciences, Mack Publishing Company, Easton, Pa., 17th edition, 1985. The composition or formulation to be administered will, in any event, contain a quantity of the compound adequate to achieve the desired state in the subject being treated.

Routes of administration applicable to the methods and compositions described herein include intranasal, intraperitoneal, intramuscular, subcutaneous, intradermal, topical application, intravenous, nasal, oral, inhalation, intralacrimal, retrolacrimal perfusion along the duct, intralacrimal, and other enteral and parenteral routes of administration. Routes of administration may be combined, if desired, or adjusted depending upon the agent and/or the desired effect. An active agent can be administered in a single dose or in multiple doses. Embodiments of these methods and routes suitable for delivery, include systemic or localized routes. In one embodiment, the composition comprising the ELP and agent is administered intralacrimally through injection. In further embodiments, the composition is administered systemically, topically on top of the eye, by retrolacrimal perfusion, or intranasally.

### Treatment of Disease

In one aspect, this disclosure provides methods and compositions useful in treating a patient in need of a blood transfusion for example, during surgery or during the treatment of trauma such as serious injuries. The fusion proteins of this disclosure also can be used to prevent extravasation and reduce nitric oxide scavenging-mediated toxicities. Thus, the method can be used to treat anemia, hemophilia, thalassemia, sickle cell anemia, or malaria by administering an effective amount of the agent as described herein to a subject or patient in need thereof. In one aspect, the agent is administered intravenously.

### Use of Compounds for Preparing Medicaments

The ELPs of the present disclosure are also useful in the preparation of medicaments to treat a variety of pathologies as described herein. The methods and techniques for preparing medicaments of a composition are known in the art. For the purpose of illustration only, pharmaceutical formulations and routes of delivery are detailed herein. In one aspect when the ELP is combined with another therapy or therapeutic agent, provided herein the compositions are useful in the preparation of combination compositions that can be simultaneously or concurrently administered.

Thus, one of skill in the art would readily appreciate that any one or more of the compositions described above, including the many specific embodiments, can be used by applying standard pharmaceutical manufacturing procedures to prepare medicaments to treat the many disorders described herein. Such medicaments can be delivered to the subject by using delivery methods known in the pharmaceutical arts.

### Kits

The ELPs as described herein, can be provided in kits. The kits can further contain additional therapeutics and optionally, instructions for making or using the ELPs. In a further aspect, the kit contains reagents and instructions to perform a screen as detailed herein.

### Screening Assays

This disclosure also provides screening assays to identify potential therapeutic agents of known and new compounds and combinations. For example, one of skill in the art can also determine if the ELP provides a therapeutic benefit in vitro by contacting the ELP or combination comprising the ELP with a sample cell or tissue to be treated. The cell or tissue can be from any species, e.g., simian, canine, bovine, ovine, rat, mouse or human.

The contacting can also be performed in vivo in an appropriate animal model or human patient. When performed in vitro, the ELPs can be directly added to the cell culture medium. When practiced in vitro, the method can be used to screen for novel combination therapies, formulations or treatment regimens, prior to administration to an animal or a human patient.

In another aspect, the assay requires contacting a first sample comprising suitable cells or tissue (“control sample”) with an effective amount of an ELP as disclosed herein and contacting a second sample of the suitable cells or tissue (“test sample”) with the ELP, agent or combination to be assayed. In one aspect in the case of cancer, the inhibition of growth of the first and second cell samples are determined. If the inhibition of growth of the second sample is substantially the same or greater than the first sample, then the agent is a potential drug for therapy. In one aspect, substantially the same or greater inhibition of growth of the cells is a difference of less than about 1%, or alternatively less than about 5% or alternatively less than about 10%, or alternatively greater than about 10%, or alternatively greater than about 20%, or alternatively greater than about 50%, or alternatively greater than about 90%. The contacting can be in vitro or in vivo. Means for determining the inhibition of growth of the cells are well known in the art.

In a further aspect, the test agent is contacted with a third sample of cells or tissue comprising normal counterpart cells or tissue to the control and test samples and selecting agents that treat the second sample of cells or tissue but does not adversely affect the third sample. For the purpose of the assays described herein, a suitable cell or tissue is described herein such as cancer or other diseases as described herein. Examples of such include, but are not limited to cancer cell or tissue obtained by biopsy, blood, breast cells, colon cells.

Efficacy of the test composition is determined using methods known in the art which include, but are not limited to cell viability assays or apoptosis evaluation.

In yet a further aspect, the assay requires at least two cell types, the first being a suitable control cell.

The assays also are useful to predict whether a subject will be suitably treated by this disclosure by delivering an ELP to a sample containing the cell to be treated and assaying for treatment, which will vary with the pathology, or for screening for new drugs and combinations. In one aspect, the cell or tissue is obtained from the subject or patient by biopsy. This disclosure also provides kits for determining whether a pathological cell or a patient will be suitably treated by this therapy by providing at least one composition of this disclosure and instructions for use.

The test cells can be grown in small multi-well plates and is used to detect the biological activity of test compounds. For the purposes of this disclosure, the successful ELP or other agent will block the growth or kill the cancer cell but leave the control cell type unharmed.

### Combination Treatments

Administration of the therapeutic agent or substance of the present disclosure to a patient will follow general protocols for the administration of that particular secondary therapy, taking into account the toxicity, if any, of the treatment. It is expected that the treatment cycles would be repeated as necessary. It also is contemplated that various standard therapies, as well as surgical intervention, may be applied in combination with the described therapy.

As is apparent to those skilled in the art, the combination therapy can take the form of a combined therapy for concurrent or sequential administration.

The following examples are included to demonstrate some embodiments of the disclosure. However, those of skill in the art should, in light of the present disclosure, appreciate that many changes can be made in the specific embodiments which are disclosed and still obtain a like or similar result without departing from the spirit and scope of the disclosure.

### Modes for Carrying Out the Disclosure

As the most common type of blood cell, red blood cells (RBCs) comprise approximately 99% of the cells in blood, which are vertebrate organism's principal means to deliver oxygen to the body tissue and remove carbon dioxide from the tissue via blood flow through the circulatory system. The reversible oxygenation function of RBCs (i.e. a large volume of oxygen taken up in the lungs and delivered to the tissues and the removal of carbon dioxide) is carried out by hemoglobin (Hb), which is the iron-containing oxygen-transport metalloprotein in the red blood cells of all vertebrates (Anthea et al., 1993) and accounts for 96% of the dry weight of the RBCs in mammals. Hb has an oxygen-binding capacity of 1.34 mL 02 per gram (DE VILLOTA et al., 1981), which increases the total blood oxygen capacity seventy-fold compared to dissolved oxygen in blood.

Similar to many other multi-subunit globular proteins, hemoglobin has a quaternary structure coming from its four subunits in roughly a tetrahedral arrangement. In most vertebrates, the hemoglobin molecule is an assembly of four globular protein subunits. Each subunit is composed of a protein chain tightly associated with an iron ion containing non-protein heme group. In adult humans, the most common hemoglobin type is a tetramer (which contains four subunit proteins) called hemoglobin A, consisting of two α and two β subunits non-covalently bound, each made of 141 and 146 amino acid residues, respectively. This is denoted as α2β2. The subunits are structurally similar and about the same size. Each subunit has a molecular weight of about 16,000 daltons, for a total molecular weight of the tetramer of about 64,000 daltons (64,458 g/mol). (Van Beekvelt et al., 2001) Hemoglobin A is the most intensively studied of the hemoglobin molecules. When oxygen binds to the first subunit of deoxyhemoglobin, the first oxygen molecule increases the affinity of the remaining subunits for additional oxygen molecules. As additional oxygen is bound to the other hemoglobin subunits, oxygen binding is incrementally strengthened, so that hemoglobin is fully oxygen-saturated at the oxygen tension of lung alveoli. Likewise, oxygen is incrementally unloaded and the affinity of hemoglobin for oxygen is reduced as oxyhemoglobin circulates to deoxygenated tissue. The absorption spectra of oxyhemoglobin and deoxyhemoglobin differ. The oxyhemoglobin has significantly lower absorption of the 660 nm wavelength than deoxyhemoglobin, while at 940 nm its absorption is slightly higher.

There are a lot of circumstances where large amounts of blood are required immediately including civilian emergency, battlefield and so on. But donated blood has its obvious limitations. First, red blood cells can only be stored for limited period. The clinical practice has established a 21-day preservation limit at 2-6° C. for red blood cell; Second, compatibility issue has to be taken into consideration when it comes to donated blood cell. Third, the source of donated blood is limited due to irregular donation.

Hemoglobin itself, although taking the responsibility of oxygen transportation, cannot be injected into circulation system directly. A preclinical model mimicking the injury during hemolytic states (Minneci et al., 2005), in which hemoglobin is released into the circulation system, along with many other similar tests (Caron et al., 1999; Gould et al., 2002; Vandegriff et al., 2003), has shown that, without being contained by a red cell membrane, free hemoglobin released into the vasculature can rapidly scavenge nitric oxide and thus lead to systemic vasoconstriction, decreased blood flow, increased release of pro-inflammatory mediators and potent vasoconstrictors and a loss of platelet inactivation (De Caterina et al., 1995; Lin et al., 2001; Rother et al., 2005), which could eventually create the conditions vascular thrombosis of the heart or other organs. Another major challenge with free hemoglobin lies in its low molecular weight, which permits rapid removal from the kidneys leading to renal toxicity.

In order to minimize such toxicities, a larger, more stable hemoglobin-based blood substitute molecules are proposed to prevent extravasation and reduce the nitric oxide scavenging-mediated toxicities. Many methods have been tried, including polymerization and pegylation of hemoglobin.

### Hemoglobin-ELP-Based Blood Substitute

The ELP conjugates are made by fusing elastin-like polypeptide (ELP), to hemoglobin though either chemical conjugation or protein engineering.

Derived from human tropoelastin, ELPs consist of pentameric repeats of (Val-Pro-Gly-Xaa-Gly)n and have a unique inverse transition behavior wherein below their transition temperature (Tt), they are highly water soluble but once the temperature rises above their Tt, ELPs undergo the phase separation process and self-assemble into particles (Dhandhukia et al., 2013). This is a fully reversible process and can be used to effectively purify ELP-conjugated materials (Shah et al., 2013). Phase behavior can be precisely controlled through adjusting the hydrophobicity of guess residue “Xaa” and number of pentapeptide repeats “n” (Urry, 1997). The ELP used in the preparation of recombinant hemoglobin is A192, with the amino acid sequence of G(VPGAG)₁₉₂Y and the molecular weight of 73.6 kDa.

ELPs are attractive as hemoglobin delivery systems for at least five important reasons: first, because ELPs can be genetically encoded, their synthesis from a synthetic gene in a heterologous host (e.g., bacteria or eukaryotic cell) can provide complete control over the amino acid sequence and molecular weight, two variables that are not easy to precisely control in synthetic polymers. Second, ELPs can be expressed from a plasmid-borne gene in E. coli to relatively high yields (500 mg/L growth), which also makes them attractive for hemoglobin delivery applications where large quantities of polymer are often required. Third, they can be purified from E. coli and other cell lysates in batch process by exploiting their inverse temperature phase transition without the need for chromatography, which simplifies large scale purification of ELPs (Meyer and Chilkoti, 1999). Fourth, ELPs can be engineered to approach the viscoelastic properties of native elastin upon crosslinking. Fifth, they are biocompatible, biodegradable, and non-immunogenic (Urry, 1997).

The versatility of ELPs have been underscored by the fact that ELPs can be appended to other proteins since 1999 by Chilkoti et al (Meyer and Chilkoti, 1999). In Dr. Mackay's lab, ELP fusions are widely investigated to treat cancer and ocular diseases (Janib et al., 2014a; Shah et al., 2013; Wang et al., 2014; Wang et al., 2015). The feasibility and strategy of expressing hemoglobin through molecular cloning have been investigated since 1985 (Nagai et al., 1985), when Nagai et al. described a method for the production and engineering of semisynthetic human Hb in E. coli. Synthesized β-globin was found to be able to refold in vitro in the presence of native α-globin and heme to gain a fully functional tetramer. Later in 1990 (Hoffman et al., 1990), Stephen et al. successfully expressed fully functional tetrameric human hemoglobin in E. coli through expressing both human α- and β-globin from a single operon at the same time. Two different globin chains can fold in vivo and incorporate endogenous heme. They also tried to express the α- and β-globin separately and found that the presence of α- and β-globin in the same cell stabilizes α-globin and aids the correct folding of β-globin.

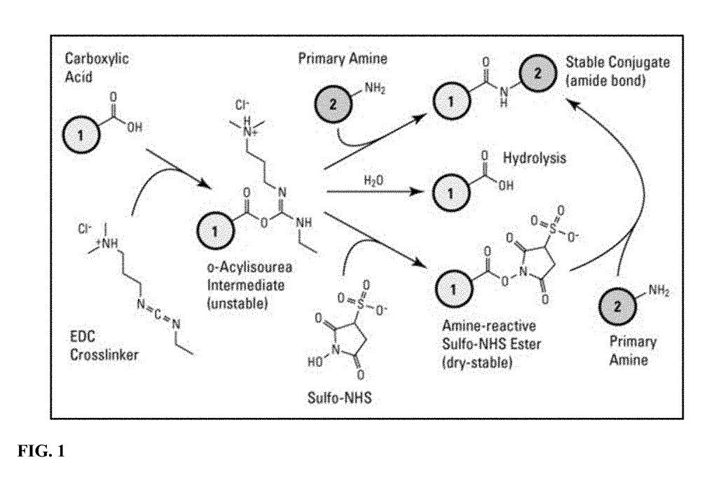

In terms of chemical conjugation, carboxyl-reactive chemical groups in bio-molecular probes are commonly used for labeling and crosslinking carboxylic acids to primary amines. For aqueous crosslinking, the most readily available and commonly used carbodiimides are the water-soluble 1-ethyl-3-(-3-dimethylaminopropyl) carbodiimide hydrochloride (EDC). EDC reacts with carboxylic acid groups to form an active O-acylisourea intermediate that is easily displaced by nucleophilic attack from primary amino groups in the reaction mixture. The primary amine forms an amide bond with the original carboxyl group, and an EDC by-product is released as a soluble urea derivative. The O-acylisourea intermediate is unstable in aqueous solutions. Thus, N-hydroxysuccinimide (NHS) or its water-soluble analog (Sulfo-NHS) is often included in EDC coupling protocols to improve efficiency or create dry-stable (amine-reactive) intermediates. EDC couples NHS to carboxyls, forming an NHS ester that is considerably more stable than the O-acylisourea intermediate while allowing for efficient conjugation to primary amines at physiologic pH (FIG. 1).

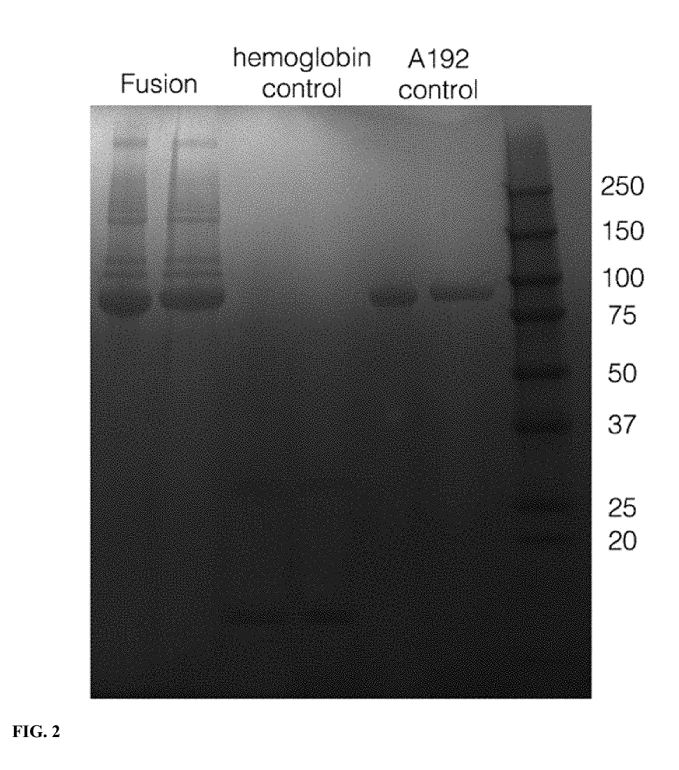

EDC-based chemical conjugation between hemoglobin subunits and A192 were fabricated, which was proved by SDS-PAGE (FIG. 2). Under reducing environment, hemoglobin tetramer was disassembled, which is why the band representing plain hemoglobin were located between 15-20 kDa. Interestingly, on lanes of fusion samples, two bands above plain A192 can be found, and without being bound by theory, is A192 binding to either one subunit or two subunits of hemoglobin.

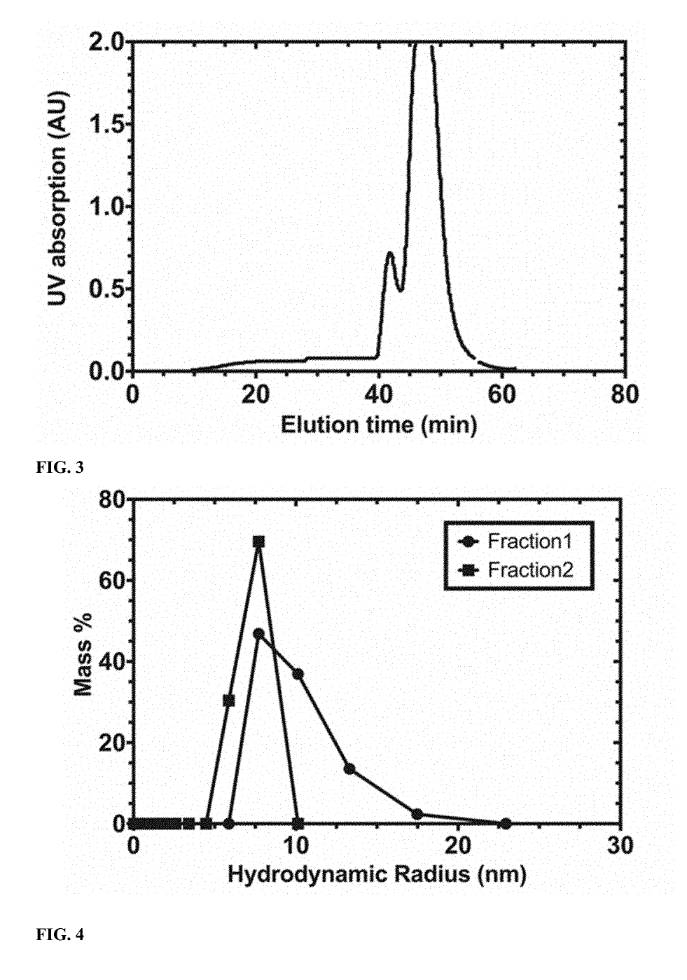

Then size exclusion chromatography (SEC) was used to separate fusion protein from plain A192 depending on their difference of molecular weight. Two peaks were observed (FIG. 3). Without being bound by theory, the first peak represents fusion protein and second peak stands for plain A192.

Those two peaks from SEC were collected separately: fraction 1 and fraction 2. Using Dynamic Light Scattering (DLS), we can measure the hydrodynamic radius of these two fractions. As shown in FIG. 4, the hydrodynamic radium of fraction 1 is 11.4 nm, significantly bigger than fraction 2, which has a radium of 7.4 nm. 7.4 nm hydrodynamic radius of fraction 2 is consistent with our previous study of A192 (Janib et al., 2014a).

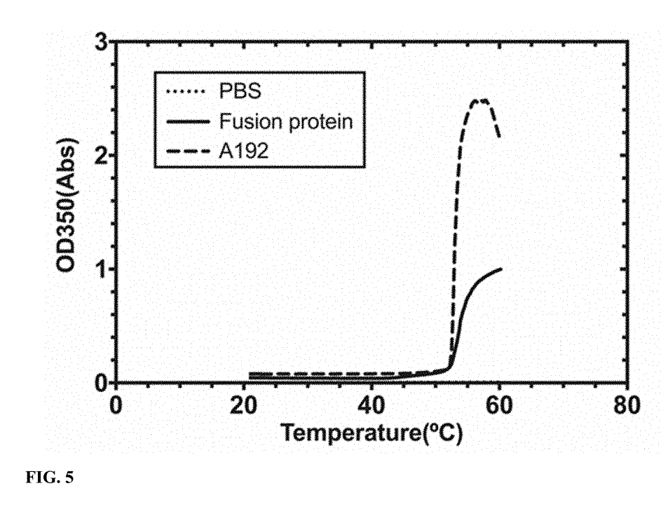

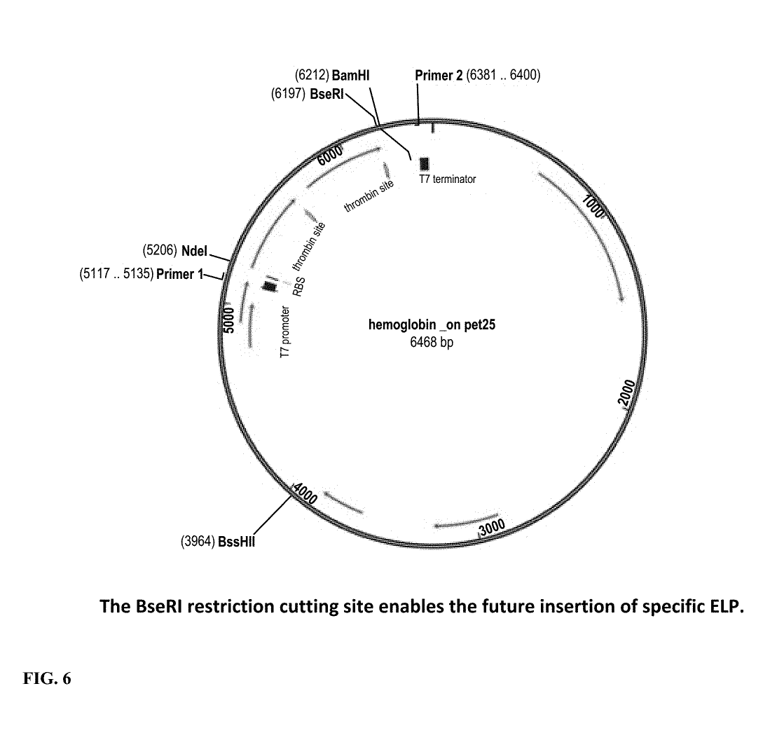

One important property of ELP is that it undergoes phase separation once the temperature reaches its transition temperature. The thermal responsiveness property of fusion protein, along with the parent A192, was characterized using Ultraviolet-visible spectroscopy (UV-Vis) by measuring their optical density of these constructs at 350 nm, where neither fusion protein nor plain A192 contributes significant absorption. As shown in FIG. 5, after conjugation, the phase separation property of ELPs is well maintained.

Although this chemical conjugation method demonstrated the feasibility of fusing hemoglobin to one of our ELPs, A192, there are too many uncontrollable factors existing in this procedure. Considering EDC-based chemical crosslinking are randomly happening in the solution, different protein combination can be achieved, which leads to the big challenge of making consistent products. In order to solve this problem, molecular cloning can be used, for example by use of an hemoglobin-ELP expressing plasmid for expression in an Escherichia coli expressing system.

Biosynthesis and purification of A192

As Previously Reported, the Gene Encoding A192 was Synthesized by Directional Ligation in a modified pET25b(+) vector (McDaniel et al., 2010). Then these reconstructed plasmids were transfected into TOP10 cells for amplification. After 18 h culture under constant shaking, a mini-preparation was contacted to obtain purified plasmids. BLR cells transfected with these purified plasmids were then introduced for A192 expression. After 24 h incubation, expressing cells were collected from the culture by centrifugation and underwent sonication to be lysed. Following that, polyethyleneimine (PEI) was used to precipitate nucleic acids from crude cell lysates. After another centrifugation, the supernatant was transferred to 50 mL tubes. All the tubes were placed on 37° C. heating block. 2M NaCl was then added into each tube. When solution turns turbid (indicates ELP phase separation), perform a Hot Spin (37° C., 12 min, 4000 rpm). After Hot Spin, keep pellet. Place those tubes containing the pellets on ice. Add 2 mL of cold PBS to re-suspend pellet. Once re-suspended, mix all re-suspended liquid into 1 tube and then perform a Cold Spin (4° C., 12 mins, 12,000 rpm). After the Cold Spin, transfer all the supernatant to one tube so that it'll be one solution. This marks one cycle of the first round of ITC (Inverse Transition Cycling). Repeat ITC at least two more times.

The ELP concentration was determined by measuring A280 by Nanodrop 2000 spectrophotometer. The molar absorption coefficient of a peptide or protein is related to its tryptophan (W), tyrosine (Y) and cysteine (C) amino acid composition (Gill and Von Hippel, 1989; Pace et al., 1995). At 280 nm, this value is approximated by the weighted sum of the 280 nm molar absorption coefficients of these three constituent amino acids, as described in the following equation:

ε=(nW×5500)+(nY×1490)+(nC×125)

where n is the number of each residue and the stated values are the amino acid molar absorptivity at 280 nm.

### Chemical Conjugation Method

Hemoglobin and A192 were first mixed at different ratios (1:1, 1:2, 1:4). Ten times molar excess of EDC as the molarity of hemoglobin was then added into the mixture. In order to achieve higher reaction efficiency, the same molarity of Sulfo-NHS as that of EDC was also added into the reaction solution. After being rotated overnight, ELP fusion, as well as plain ELP, was purified by another ITC from the mixture.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

Sequence Listing

A192: G(VPGAG)₁₉₂Y
A96I96: G(VPGAG)₉₆(VPGIG)₉₆Y
A96: G(VPGAG)₉₆Y
Hemoglobin-ELP amino-acid fusion sequences that we
intend to prepare and characterize:
Alpha-A192:
MVLSPADKTNVKAAWGKVGAHAGEYGAEALERMFLSFPTTKTYFPHFDLS
HGSAQVKGHGKKVADALTNAVAHVDDMPNALSALSDLHAHKLRVDPVNFK
LLSHCLLVTLAAHLPAEFTPAVHASLDKFLASVSTVLTSKYR-GLVPRGS
GH- G(VPGAG)₁₉₂Y
Beta-A192:
MVHLTPEEKSAVTALWGKVNVDEVGGEALGRLLVVYPWTQRFFESFGDLS
TPDAVMGNPKVKAHGKKVLGAFSDGLAHLDNLKGTFATLSELHCDKLHVD
PENFRLLGNVLVCVLAHHFGKEFTPPVQAAYQKVVAGVANALAHKYH-GL
VPRGSGH- G(VPGAG)₁₉₂Y
Alpha-A96I96:
MVLSPADKTNVKAAWGKVGAHAGEYGAEALERMFLSFPTTKTYFPHFDLS
HGSAQVKGHGKKVADALTNAVAHVDDMPNALSALSDLHAHKLRVDPVNFK
LLSHCLLVTLAAHLPAEFTPAVHASLDKFLASVSTVLTSKYR-GLVPRGS
GH- G(VPGAG)₉₆(VPGIG)₉₆Y
Beta-A96I96:
MVHLTPEEKSAVTALWGKVNVDEVGGEALGRLLVVYPWTQRFFESFGDLS
TPDAVMGNPKVKAHGKKVLGAFSDGLAHLDNLKGTFATLSELHCDKLHVD
PENFRLLGNVLVCVLAHHFGKEFTPPVQAAYQKVVAGVANALAHKYH-GL
VPRGSGH- G(VPGAG)₉₆(VPGIG)₉₆Y
Alpha-A96:
MVLSPADKTNVKAAWGKVGAHAGEYGAEALERMFLSFPTTKTYFPHFDLS
HGSAQVKGHGKKVADALTNAVAHVDDMPNALSALSDLHAHKLRVDPVNFK
LLSHCLLVTLAAHLPAEFTPAVHASLDKFLASVSTVLTSKYR-GLVPRGS
GH- G(VPGAG)₉₆Y
Beta-A96:
MVHLTPEEKSAVTALWGKVNVDEVGGEALGRLLVVYPWTQRFFESFGDLS
TPDAVMGNPKVKAHGKKVLGAFSDGLAHLDNLKGTFATLSELHCDKLHVD
PENFRLLGNVLVCVLAHHFGKEFTPPVQAAYQKVVAGVANALAHKYH-GL
VPRGSGH- G(VPGAG)₉₆Y
Canis lupus familiaris (Dog):
Alpha:
MVLSPADKTNIKSTWDKIGGHAGDYGGEALDRTFQSFPTTKTYFPHFDLS
PGSAQVKAHGKKVADALTTAVAHLDDLPGALSALSDLHAYKLRVDPVNFK
LLSHCLLVTLACHHPTEFTPAVHASLDKFFAAVSTVLTSKYR
Beta:
MVHLTAEEKSLVSGLWGKVNVDEVGGEALGRLLIVYPWTQRFFDSFGDLS
TPDAVMSNAKVKAHGKKVLNSFSDGLKNLDNLKGTFAKLSELHCDKLHVD
PENFKLLGNVLVCVLAHHFGKEFTPQVQAAYQKVVAGVANALAHKYH
Felis catus (Cat):
Alpha:
MVLSAADKSNVKACWGKIGSHAGEYGAEALERTFCSFPTTKTYFPHFDLS
HGSAQVKAHGQKVADALTQAVAHMDDLPTAMSALSDLHAYKLRVDPVNFK
FLSHCLLVTLACHHPAEFTPAVHASLDKFFSAVSTVLTSKYR
Beta:
MGFLSAEEKGMVNGLWGKVNVDEVGGEALGRLLVVYPWTQRFFQSFGDLS
SADAIMSNSKVKAHGKKVLNSFSDGLKNIDDLKGAFAKLSELHCDKLHVD
PENFRLLGNVLVCVLAHHFGHDFNPQVQAAFQKVVAGVANALAHKYH
Equus caballus (horse):
Alpha:
MVLSAADKTNVKAAWSKVGGHAGEFGAEALERMFLGFPTTKTYFPHFDLS
HGSAQVKAHGKKVGDALTLAVGHLDDLPGALSNLSDLHAHKLRVDPVNFK
LLSHCLLSTLAVHLPNDFTPAVHASLDKFLSSVSTVLTSKYR
Beta:
MVQLSGEEKAAVLALWDKVNEEEVGGEALGRLLVVYPWTQRFFDSFGDLS
NPGAVMGNPKVKAHGKKVLHSFGEGVHHLDNLKGTFAALSELHCDKLHVD
PENFRLLGNVLVVVLARHFGKDFTPELQASYQKVVAGVANALAHKYH

### REFERENCES

1. Anthea, M., Hopkins, J., McLaughlin, C. W., Johnson, S., Warner, M. Q., LaHart, D., and Wright, J. D. (1993). Human Biology and Health. Englewood Cliffs, N.J., USA: Pentice Hall (ISBN 0-13-981176-1. OCLC 32308337).
2. Caron, A., Menu, P., Faivre-Fiorina, B., Labrude, P., Alayash, A. I., and Vigneron, C. (1999). Cardiovascular and hemorheological effects of three modified human hemoglobin solutions in hemodiluted rabbits. Journal of Applied Physiology 86, 541-548.
3. De Caterina, R., Libby, P., Peng, H.-B., Thannickal, V. J., Rajavashisth, T., Gimbrone Jr, M., Shin, W. S., and Liao, J. K. (1995). Nitric oxide decreases cytokine-induced endothelial activation. Nitric oxide selectively reduces endothelial expression of adhesion molecules and proinflammatory cytokines. Journal of Clinical Investigation 96, 60.
4. DE VILLOTA, E. D., CARMONA, M. G., Rubio, J., and DE ANDRES, S. R. (1981). Equality of the in vivo and in vitro oxygen-binding capacity of haemoglobin in patients with severe respiratory disease. British Journal of Anaesthesia 53, 1325-1328.
5. Dhandhukia, J., Weitzhandler, I., Wang, W., and MacKay, J. A. (2013). Switchable elastin-like polypeptides that respond to chemical inducers of dimerization. Biomacromolecules 14, 976-985.
6. Gill, S. C., and Von Hippel, P. H. (1989). Calculation of protein extinction coefficients from amino acid sequence data. Analytical biochemistry 182, 319-326.
7. Gould, S. A., Moore, E. E., Hoyt, D. B., Ness, P. M., Norris, E. J., Carson, J. L., Hides, G. A., Freeman, I. H., DeWoskin, R., and Moss, G. S. (2002). The life-sustaining capacity of human polymerized hemoglobin when red cells might be unavailable. Journal of the American College of Surgeons 195, 445-452.
8. Hoffman, S. J., Looker, D. L., Roehrich, J. M., Cozart, P. E., Durfee, S. L., Tedesco, J. L., and Stetler, G. L. (1990). Expression of fully functional tetrameric human hemoglobin in Escherichia coli. Proceedings of the National Academy of Sciences 87, 8521-8525.
9. Janib, S. M., Gustafson, J. A., Minea, R. O., Swenson, S. D., Liu, S., Pastuszka, M. K., Lock, L. L., Cui, H., Markland, F. S., and Conti, P. S. (2014). Multimeric disintegrin protein polymer fusions that target tumor vasculature. Biomacromolecules 15, 2347-2358.
10. Lin, G., Macdonald, R. L., Marton, L. S., Kowalczuk, A., Solenski, N. J., and Weir, B. K. (2001). Hemoglobin increases endothelin-1 in endothelial cells by decreasing nitric oxide. Biochemical and biophysical research communications 280, 824-830.
11. McDaniel, J. R., MacKay, J. A., Quiroz, F. G., and Chilkoti, A. (2010). Recursive directional ligation by plasmid reconstruction allows rapid and seamless cloning of oligomeric genes. Biomacromolecules 11, 944-952.
12. Meyer, D. E., and Chilkoti, A. (1999). Purification of recombinant proteins by fusion with thermally-responsive polypeptides. Nature biotechnology 17, 1112-1115.
13. Minneci, P. C., Deans, K. J., Zhi, H., Yuen, P. S., Star, R. A., Banks, S. M., Schechter, A. N., Natanson, C., Gladwin, M. T., and Solomon, S. B. (2005). Hemolysis-associated endothelial dysfunction mediated by accelerated NO inactivation by decompartmentalized oxyhemoglobin. The Journal of clinical investigation 115, 3409-3417.
14. Nagai, K., Perutz, M. F., and Poyart, C. (1985). Oxygen binding properties of human mutant hemoglobins synthesized in Escherichia coli. Proceedings of the National Academy of Sciences 82, 7252-7255.
15. Pace, C. N., Vajdos, F., Fee, L., Grimsley, G., and Gray, T. (1995). How to measure and predict the molar absorption coefficient of a protein. Protein science 4, 2411-2423.
16. Rother, R. P., Bell, L., Hillmen, P., and Gladwin, M. T. (2005). The clinical sequelae of intravascular hemolysis and extracellular plasma hemoglobin: a novel mechanism of human disease. Jama 293, 1653-1662.
17. Shah, M., Edman, M. C., Janga, S. R., Shi, P., Dhandhukia, J., Liu, S., Louie, S. G., Rodgers, K., MacKay, J. A., and Hamm-Alvarez, S. F. (2013). A rapamycin-binding protein polymer nanoparticle shows potent therapeutic activity in suppressing autoimmune dacryoadenitis in a mouse model of Sjögren's syndrome. Journal of Controlled Release 171, 269-279.
18. Urry, D. W. (1997). Physical chemistry of biological free energy transduction as demonstrated by elastic protein-based polymers. The Journal of Physical Chemistry B 101, 11007-11028.
19. Van Beekvelt, M. C., Colier, W., Wevers, R. A., and Van Engelen, B. G. (2001). Performance of near-infrared spectroscopy in measuring local O˜ 2 consumption and blood flow in skeletal muscle. Journal of Applied Physiology 90, 511-519.
20. Vandegriff, K. D., Malavalli, A., Wooldridge, J., Lohman, J., and Winslow, R. M. (2003). MP4, a new nonvasoactive PEG-Hb conjugate. Transfusion 43, 509-516.
21. Wang, W., Despanie, J., Shi, P., Edman, M. C., Lin, Y.-A., Cui, H., Heur, M., Fini, M. E., Hamm-Alvarez, S. F., and MacKay, J. A. (2014). Lacritin-mediated regeneration of the corneal epithelia by protein polymer nanoparticles. Journal of Materials Chemistry B 2, 8131-8141.
22. Wang, W., Jashnani, A., Aluri, S. R., Gustafson, J. A., Hsueh, P.-Y., Yarber, F., McKown, R. L., Laurie, G. W., Hamm-Alvarez, S. F., and MacKay, J. A. (2015). A thermo-responsive protein treatment for dry eyes. Journal of Controlled Release 199, 156-167.

1. An agent comprising an elastin-like peptide (ELP) component fused to a hemoglobin protein, or a fragment of the hemoglobin protein.

2. The agent of claim 1, wherein the hemoglobin fragment is an alpha or beta subunit of hemoglobin.

3. The agent of claim 1, wherein the hemoglobin protein comprises the alpha and beta subunit of hemoglobin.

4. The agent of claim 1, wherein the ELP comprises a reference polypeptide (VPGXG)n (SEQ ID NO: 1), (wherein n is an integer that denotes the number of repeats, and can be from about between 5 and 400, alternatively between 5 and 300, or alternatively between 25 and 250, or alternatively between 25 and 150, or from about 6 to about 200, or alternatively from about 15 to 195, or alternatively from 40 to about 195, or alternatively about 24, or alternatively about 48, or alternatively about 96, or alternatively about 192, and X is an amino acid selected from Ser, Ala, Ile, or Val, or a biological equivalent thereof, wherein a biological equivalent of the reference polypeptide is a peptide that has at least 80% sequence identity to the reference polypeptide or a peptide encoded by a polynucleotide that hybridizes under conditions of high stringency to a polynucleotide that encodes the reference polypeptide or its complement, wherein conditions of high stringency comprise hybridization reaction at about 60° C. in about 1×SSC.

5. The agent of claim 1, wherein the ELP comprises A192 or a biological equivalent thereof, wherein a biological equivalent of the reference polypeptide is a peptide that has at least 80% sequence identity to the reference polypeptide or a peptide encoded by a polynucleotide that hybridizes under conditions of high stringency to a polynucleotide that encodes the reference polypeptide or its complement, wherein conditions of high stringency comprise hybridization reaction at about 60° C. in about 1×SSC.

6. The agent of claim 1, wherein the ELP consists of A192 or a biological equivalent thereof, wherein a biological equivalent of the reference polypeptide is a peptide that has at least 80% sequence identity to the reference polypeptide or a peptide encoded by a polynucleotide that hybridizes under conditions of high stringency to a polynucleotide that encodes the reference polypeptide or its complement, wherein conditions of high stringency comprise hybridization reaction at about 60° C. in about 1×SSC.

7. An isolated polynucleotide encoding the agent of claim 1.

8. A vector comprising the isolated polynucleotide of claim 7.

9. The vector of claim 8, wherein the vector is a plasmid.

10. The vector of claim 9, wherein the plasmid is hemoglobin_on pet25.

11. A host cell comprising the isolated polynucleotide of claim 7.

12. A host cell comprising the vector of claim 8.

13. The host cell of claim 11, wherein the host cell is a eukaryotic cell or a prokaryotic cell.

14. The host cell of claim 13, wherein the prokaryotic cell is an E. coli cell.

15. A method for preparing an agent, comprising growing the host cell of claim 11 under conditions that favor expression of the polynucleotide.

16. The method of claim 15, further comprising isolating the agent prepared by the method of claim 15.

17. A composition comprising the agent of claim 1 and a carrier.

18. A composition comprising the isolated polynucleotide of claim 7 and a carrier.

19. The composition of claim 17 or 18, wherein the carrier is a pharmaceutically acceptable carrier.

20. A method to treat a patient in need thereof, comprising administering an effective amount of the agent of claim 1, thereby treating the patient.

21. The method of claim 20, wherein the patient is in need of a blood transfusion.

22. The method of claim 20, wherein the patient is a mammal.

23. The method of claim 22, wherein the mammal is a human.

24. An ELP fusion comprising an ELP polypeptide fused to a hemoglobin polypeptide or a fragment of the hemoglobin polypeptide.

25. An isolated polynucleotide encoding the ELP fusion of claim 24.

26. A composition comprising the ELP fusion of claim 24.

27. A method for treating a subject in need of a blood transfusion comprising administering an effective amount of the ELP fusion of claim 24 to the subject.

28. The method of claim 27, wherein the subject in need of a blood transfusion is suffering from a condition from the group of anemia, hemophilia, thalassemia, sickle cell anemia, or malaria.

29. A method for preventing extravasation and reducing nitric oxide scavenging-mediated toxicities in a patient in need thereof, comprising administering an effective amount of the ELP fusion of claim 1 to the subject.


SEQUENCE LISTING (20 sequences)

SEQ ID NO: 1 - protein, 5 aa, Artificial Sequence
  Description of Artificial Sequence Synthetic peptide
  MOD_RES (4)..(4): Ser, Ala, Ile, or Val, or a biological equivalent thereof
  See specification as filed for detailed description of substitutions and preferred embodiments
VPGXG

SEQ ID NO: 2 - protein, 5 aa, Artificial Sequence
  Description of Artificial Sequence Synthetic peptide
  MOD_RES (4)..(4): Any amino acid
  See specification as filed for detailed description of substitutions and preferred embodiments
VPGXG

SEQ ID NO: 3 - protein, 962 aa, Artificial Sequence
  Description of Artificial Sequence Synthetic polypeptide
GVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGY

SEQ ID NO: 4 - protein, 482 aa, Artificial Sequence
  Description of Artificial Sequence Synthetic polypeptide
GVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGY

SEQ ID NO: 5 - protein, 12 aa, Artificial Sequence
  Description of Artificial Sequence Synthetic peptide
  See specification as filed for detailed description of substitutions and preferred embodiments
GVPGAGVPGIGY

SEQ ID NO: 6 - protein, 962 aa, Artificial Sequence
  Description of Artificial Sequence Synthetic polypeptide
GVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGY

SEQ ID NO: 7 - protein, 5 aa, Artificial Sequence
  Description of Artificial Sequence Synthetic peptide
  MOD_RES (4)..(4): Any amino acid, an amino acid analog or amino acid derivative thereof
  See specification as filed for detailed description of substitutions and preferred embodiments
VPGXG

SEQ ID NO: 8 - protein, 2000 aa, Artificial Sequence
  Description of Artificial Sequence Synthetic polypeptide
  MISC_FEATURE (1)..(2000): This sequence may encompass 5 to 400 “Val Pro Gly Xaa Gly” repeating units
  MOD_RES (4)..(4): Any amino acid
  MOD_RES (9)..(9): Any amino acid
  MOD_RES (14)..(14): Any amino acid
  MOD_RES (19)..(19): Any amino acid
  MOD_RES (24)..(24): Any amino acid
  MOD_RES (29)..(29): Any amino acid
  MOD_RES (34)..(34): Any amino acid
  MOD_RES (39)..(39): Any amino acid
  MOD_RES (44)..(44): Any amino acid
  MOD_RES (49)..(49): Any amino acid
  MOD_RES (54)..(54): Any amino acid
  MOD_RES (59)..(59): Any amino acid
  MOD_RES (64)..(64): Any amino acid
  MOD_RES (69)..(69): Any amino acid
  MOD_RES (74)..(74): Any amino acid
  MOD_RES (79)..(79): Any amino acid
  MOD_RES (84)..(84): Any amino acid
  MOD_RES (89)..(89): Any amino acid
  MOD_RES (94)..(94): Any amino acid
  MOD_RES (99)..(99): Any amino acid
  MOD_RES (104)..(104): Any amino acid
  MOD_RES (109)..(109): Any amino acid
  MOD_RES (114)..(114): Any amino acid
  MOD_RES (119)..(119): Any amino acid
  MOD_RES (124)..(124): Any amino acid
  MOD_RES (129)..(129): Any amino acid
  MOD_RES (134)..(134): Any amino acid
  MOD_RES (139)..(139): Any amino acid
  MOD_RES (144)..(144): Any amino acid
  MOD_RES (149)..(149): Any amino acid
  MOD_RES (154)..(154): Any amino acid
  MOD_RES (159)..(159): Any amino acid
  MOD_RES (164)..(164): Any amino acid
  MOD_RES (169)..(169): Any amino acid
  MOD_RES (174)..(174): Any amino acid
  MOD_RES (179)..(179): Any amino acid
  MOD_RES (184)..(184): Any amino acid
  MOD_RES (189)..(189): Any amino acid
  MOD_RES (194)..(194): Any amino acid
  MOD_RES (199)..(199): Any amino acid
  MOD_RES (204)..(204): Any amino acid
  MOD_RES (209)..(209): Any amino acid
  MOD_RES (214)..(214): Any amino acid
  MOD_RES (219)..(219): Any amino acid
  MOD_RES (224)..(224): Any amino acid
  MOD_RES (229)..(229): Any amino acid
  MOD_RES (234)..(234): Any amino acid
  MOD_RES (239)..(239): Any amino acid
  MOD_RES (244)..(244): Any amino acid
  MOD_RES (249)..(249): Any amino acid
  MOD_RES (254)..(254): Any amino acid
  MOD_RES (259)..(259): Any amino acid
  MOD_RES (264)..(264): Any amino acid
  MOD_RES (269)..(269): Any amino acid
  MOD_RES (274)..(274): Any amino acid
  MOD_RES (279)..(279): Any amino acid
  MOD_RES (284)..(284): Any amino acid
  MOD_RES (289)..(289): Any amino acid
  MOD_RES (294)..(294): Any amino acid
  MOD_RES (299)..(299): Any amino acid
  MOD_RES (304)..(304): Any amino acid
  MOD_RES (309)..(309): Any amino acid
  MOD_RES (314)..(314): Any amino acid
  MOD_RES (319)..(319): Any amino acid
  MOD_RES (324)..(324): Any amino acid
  MOD_RES (329)..(329): Any amino acid
  MOD_RES (334)..(334): Any amino acid
  MOD_RES (339)..(339): Any amino acid
  MOD_RES (344)..(344): Any amino acid
  MOD_RES (349)..(349): Any amino acid
  MOD_RES (354)..(354): Any amino acid
  MOD_RES (359)..(359): Any amino acid
  MOD_RES (364)..(364): Any amino acid
  MOD_RES (369)..(369): Any amino acid
  MOD_RES (374)..(374): Any amino acid
  MOD_RES (379)..(379): Any amino acid
  MOD_RES (384)..(384): Any amino acid
  MOD_RES (389)..(389): Any amino acid
  MOD_RES (394)..(394): Any amino acid
  MOD_RES (399)..(399): Any amino acid
  MOD_RES (404)..(404): Any amino acid
  MOD_RES (409)..(409): Any amino acid
  MOD_RES (414)..(414): Any amino acid
  MOD_RES (419)..(419): Any amino acid
  MOD_RES (424)..(424): Any amino acid
  MOD_RES (429)..(429): Any amino acid
  MOD_RES (434)..(434): Any amino acid
  MOD_RES (439)..(439): Any amino acid
  MOD_RES (444)..(444): Any amino acid
  MOD_RES (449)..(449): Any amino acid
  MOD_RES (454)..(454): Any amino acid
  MOD_RES (459)..(459): Any amino acid
  MOD_RES (464)..(464): Any amino acid
  MOD_RES (469)..(469): Any amino acid
  MOD_RES (474)..(474): Any amino acid
  MOD_RES (479)..(479): Any amino acid
  MOD_RES (484)..(484): Any amino acid
  MOD_RES (489)..(489): Any amino acid
  MOD_RES (494)..(494): Any amino acid
  MOD_RES (499)..(499): Any amino acid
  MOD_RES (504)..(504): Any amino acid
  MOD_RES (509)..(509): Any amino acid
  MOD_RES (514)..(514): Any amino acid
  MOD_RES (519)..(519): Any amino acid
  MOD_RES (524)..(524): Any amino acid
  MOD_RES (529)..(529): Any amino acid
  MOD_RES (534)..(534): Any amino acid
  MOD_RES (539)..(539): Any amino acid
  MOD_RES (544)..(544): Any amino acid
  MOD_RES (549)..(549): Any amino acid
  MOD_RES (554)..(554): Any amino acid
  MOD_RES (559)..(559): Any amino acid
  MOD_RES (564)..(564): Any amino acid
  MOD_RES (569)..(569): Any amino acid
  MOD_RES (574)..(574): Any amino acid
  MOD_RES (579)..(579): Any amino acid
  MOD_RES (584)..(584): Any amino acid
  MOD_RES (589)..(589): Any amino acid
  MOD_RES (594)..(594): Any amino acid
  MOD_RES (599)..(599): Any amino acid
  MOD_RES (604)..(604): Any amino acid
  MOD_RES (609)..(609): Any amino acid
  MOD_RES (614)..(614): Any amino acid
  MOD_RES (619)..(619): Any amino acid
  MOD_RES (624)..(624): Any amino acid
  MOD_RES (629)..(629): Any amino acid
  MOD_RES (634)..(634): Any amino acid
  MOD_RES (639)..(639): Any amino acid
  MOD_RES (644)..(644): Any amino acid
  MOD_RES (649)..(649): Any amino acid
  MOD_RES (654)..(654): Any amino acid
  MOD_RES (659)..(659): Any amino acid
  MOD_RES (664)..(664): Any amino acid
  MOD_RES (669)..(669): Any amino acid
  MOD_RES (674)..(674): Any amino acid
  MOD_RES (679)..(679): Any amino acid
  MOD_RES (684)..(684): Any amino acid
  MOD_RES (689)..(689): Any amino acid
  MOD_RES (694)..(694): Any amino acid
  MOD_RES (699)..(699): Any amino acid
  MOD_RES (704)..(704): Any amino acid
  MOD_RES (709)..(709): Any amino acid
  MOD_RES (714)..(714): Any amino acid
  MOD_RES (719)..(719): Any amino acid
  MOD_RES (724)..(724): Any amino acid
  MOD_RES (729)..(729): Any amino acid
  MOD_RES (734)..(734): Any amino acid
  MOD_RES (739)..(739): Any amino acid
  MOD_RES (744)..(744): Any amino acid
  MOD_RES (749)..(749): Any amino acid
  MOD_RES (754)..(754): Any amino acid
  MOD_RES (759)..(759): Any amino acid
  MOD_RES (764)..(764): Any amino acid
  MOD_RES (769)..(769): Any amino acid
  MOD_RES (774)..(774): Any amino acid
  MOD_RES (779)..(779): Any amino acid
  MOD_RES (784)..(784): Any amino acid
  MOD_RES (789)..(789): Any amino acid
  MOD_RES (794)..(794): Any amino acid
  MOD_RES (799)..(799): Any amino acid
  MOD_RES (804)..(804): Any amino acid
  MOD_RES (809)..(809): Any amino acid
  MOD_RES (814)..(814): Any amino acid
  MOD_RES (819)..(819): Any amino acid
  MOD_RES (824)..(824): Any amino acid
  MOD_RES (829)..(829): Any amino acid
  MOD_RES (834)..(834): Any amino acid
  MOD_RES (839)..(839): Any amino acid
  MOD_RES (844)..(844): Any amino acid
  MOD_RES (849)..(849): Any amino acid
  MOD_RES (854)..(854): Any amino acid
  MOD_RES (859)..(859): Any amino acid
  MOD_RES (864)..(864): Any amino acid
  MOD_RES (869)..(869): Any amino acid
  MOD_RES (874)..(874): Any amino acid
  MOD_RES (879)..(879): Any amino acid
  MOD_RES (884)..(884): Any amino acid
  MOD_RES (889)..(889): Any amino acid
  MOD_RES (894)..(894): Any amino acid
  MOD_RES (899)..(899): Any amino acid
  MOD_RES (904)..(904): Any amino acid
  MOD_RES (909)..(909): Any amino acid
  MOD_RES (914)..(914): Any amino acid
  MOD_RES (919)..(919): Any amino acid
  MOD_RES (924)..(924): Any amino acid
  MOD_RES (929)..(929): Any amino acid
  MOD_RES (934)..(934): Any amino acid
  MOD_RES (939)..(939): Any amino acid
  MOD_RES (944)..(944): Any amino acid
  MOD_RES (949)..(949): Any amino acid
  MOD_RES (954)..(954): Any amino acid
  MOD_RES (959)..(959): Any amino acid
  MOD_RES (964)..(964): Any amino acid
  MOD_RES (969)..(969): Any amino acid
  MOD_RES (974)..(974): Any amino acid
  MOD_RES (979)..(979): Any amino acid
  MOD_RES (984)..(984): Any amino acid
  MOD_RES (989)..(989): Any amino acid
  MOD_RES (994)..(994): Any amino acid
  MOD_RES (999)..(999): Any amino acid
  MOD_RES (1004)..(1004): Any amino acid
  MOD_RES (1009)..(1009): Any amino acid
  MOD_RES (1014)..(1014): Any amino acid
  MOD_RES (1019)..(1019): Any amino acid
  MOD_RES (1024)..(1024): Any amino acid
  MOD_RES (1029)..(1029): Any amino acid
  MOD_RES (1034)..(1034): Any amino acid
  MOD_RES (1039)..(1039): Any amino acid
  MOD_RES (1044)..(1044): Any amino acid
  MOD_RES (1049)..(1049): Any amino acid
  MOD_RES (1054)..(1054): Any amino acid
  MOD_RES (1059)..(1059): Any amino acid
  MOD_RES (1064)..(1064): Any amino acid
  MOD_RES (1069)..(1069): Any amino acid
  MOD_RES (1074)..(1074): Any amino acid
  MOD_RES (1079)..(1079): Any amino acid
  MOD_RES (1084)..(1084): Any amino acid
  MOD_RES (1089)..(1089): Any amino acid
  MOD_RES (1094)..(1094): Any amino acid
  MOD_RES (1099)..(1099): Any amino acid
  MOD_RES (1104)..(1104): Any amino acid
  MOD_RES (1109)..(1109): Any amino acid
  MOD_RES (1114)..(1114): Any amino acid
  MOD_RES (1119)..(1119): Any amino acid
  MOD_RES (1124)..(1124): Any amino acid
  MOD_RES (1129)..(1129): Any amino acid
  MOD_RES (1134)..(1134): Any amino acid
  MOD_RES (1139)..(1139): Any amino acid
  MOD_RES (1144)..(1144): Any amino acid
  MOD_RES (1149)..(1149): Any amino acid
  MOD_RES (1154)..(1154): Any amino acid
  MOD_RES (1159)..(1159): Any amino acid
  MOD_RES (1164)..(1164): Any amino acid
  MOD_RES (1169)..(1169): Any amino acid
  MOD_RES (1174)..(1174): Any amino acid
  MOD_RES (1179)..(1179): Any amino acid
  MOD_RES (1184)..(1184): Any amino acid
  MOD_RES (1189)..(1189): Any amino acid
  MOD_RES (1194)..(1194): Any amino acid
  MOD_RES (1199)..(1199): Any amino acid
  MOD_RES (1204)..(1204): Any amino acid
  MOD_RES (1209)..(1209): Any amino acid
  MOD_RES (1214)..(1214): Any amino acid
  MOD_RES (1219)..(1219): Any amino acid
  MOD_RES (1224)..(1224): Any amino acid
  MOD_RES (1229)..(1229): Any amino acid
  MOD_RES (1234)..(1234): Any amino acid
  MOD_RES (1239)..(1239): Any amino acid
  MOD_RES (1244)..(1244): Any amino acid
  MOD_RES (1249)..(1249): Any amino acid
  MOD_RES (1254)..(1254): Any amino acid
  MOD_RES (1259)..(1259): Any amino acid
  MOD_RES (1264)..(1264): Any amino acid
  MOD_RES (1269)..(1269): Any amino acid
  MOD_RES (1274)..(1274): Any amino acid
  MOD_RES (1279)..(1279): Any amino acid
  MOD_RES (1284)..(1284): Any amino acid
  MOD_RES (1289)..(1289): Any amino acid
  MOD_RES (1294)..(1294): Any amino acid
  MOD_RES (1299)..(1299): Any amino acid
  MOD_RES (1304)..(1304): Any amino acid
  MOD_RES (1309)..(1309): Any amino acid
  MOD_RES (1314)..(1314): Any amino acid
  MOD_RES (1319)..(1319): Any amino acid
  MOD_RES (1324)..(1324): Any amino acid
  MOD_RES (1329)..(1329): Any amino acid
  MOD_RES (1334)..(1334): Any amino acid
  MOD_RES (1339)..(1339): Any amino acid
  MOD_RES (1344)..(1344): Any amino acid
  MOD_RES (1349)..(1349): Any amino acid
  MOD_RES (1354)..(1354): Any amino acid
  MOD_RES (1359)..(1359): Any amino acid
  MOD_RES (1364)..(1364): Any amino acid
  MOD_RES (1369)..(1369): Any amino acid
  MOD_RES (1374)..(1374): Any amino acid
  MOD_RES (1379)..(1379): Any amino acid
  MOD_RES (1384)..(1384): Any amino acid
  MOD_RES (1389)..(1389): Any amino acid
  MOD_RES (1394)..(1394): Any amino acid
  MOD_RES (1399)..(1399): Any amino acid
  MOD_RES (1404)..(1404): Any amino acid
  MOD_RES (1409)..(1409): Any amino acid
  MOD_RES (1414)..(1414): Any amino acid
  MOD_RES (1419)..(1419): Any amino acid
  MOD_RES (1424)..(1424): Any amino acid
  MOD_RES (1429)..(1429): Any amino acid
  MOD_RES (1434)..(1434): Any amino acid
  MOD_RES (1439)..(1439): Any amino acid
  MOD_RES (1444)..(1444): Any amino acid
  MOD_RES (1449)..(1449): Any amino acid
  MOD_RES (1454)..(1454): Any amino acid
  MOD_RES (1459)..(1459): Any amino acid
  MOD_RES (1464)..(1464): Any amino acid
  MOD_RES (1469)..(1469): Any amino acid
  MOD_RES (1474)..(1474): Any amino acid
  MOD_RES (1479)..(1479): Any amino acid
  MOD_RES (1484)..(1484): Any amino acid
  MOD_RES (1489)..(1489): Any amino acid
  MOD_RES (1494)..(1494): Any amino acid
  MOD_RES (1499)..(1499): Any amino acid
  MOD_RES (1504)..(1504): Any amino acid
  MOD_RES (1509)..(1509): Any amino acid
  MOD_RES (1514)..(1514): Any amino acid
  MOD_RES (1519)..(1519): Any amino acid
  MOD_RES (1524)..(1524): Any amino acid
  MOD_RES (1529)..(1529): Any amino acid
  MOD_RES (1534)..(1534): Any amino acid
  MOD_RES (1539)..(1539): Any amino acid
  MOD_RES (1544)..(1544): Any amino acid
  MOD_RES (1549)..(1549): Any amino acid
  MOD_RES (1554)..(1554): Any amino acid
  MOD_RES (1559)..(1559): Any amino acid
  MOD_RES (1564)..(1564): Any amino acid
  MOD_RES (1569)..(1569): Any amino acid
  MOD_RES (1574)..(1574): Any amino acid
  MOD_RES (1579)..(1579): Any amino acid
  MOD_RES (1584)..(1584): Any amino acid
  MOD_RES (1589)..(1589): Any amino acid
  MOD_RES (1594)..(1594): Any amino acid
  MOD_RES (1599)..(1599): Any amino acid
  MOD_RES (1604)..(1604): Any amino acid
  MOD_RES (1609)..(1609): Any amino acid
  MOD_RES (1614)..(1614): Any amino acid
  MOD_RES (1619)..(1619): Any amino acid
  MOD_RES (1624)..(1624): Any amino acid
  MOD_RES (1629)..(1629): Any amino acid
  MOD_RES (1634)..(1634): Any amino acid
  MOD_RES (1639)..(1639): Any amino acid
  MOD_RES (1644)..(1644): Any amino acid
  MOD_RES (1649)..(1649): Any amino acid
  MOD_RES (1654)..(1654): Any amino acid
  MOD_RES (1659)..(1659): Any amino acid
  MOD_RES (1664)..(1664): Any amino acid
  MOD_RES (1669)..(1669): Any amino acid
  MOD_RES (1674)..(1674): Any amino acid
  MOD_RES (1679)..(1679): Any amino acid
  MOD_RES (1684)..(1684): Any amino acid
  MOD_RES (1689)..(1689): Any amino acid
  MOD_RES (1694)..(1694): Any amino acid
  MOD_RES (1699)..(1699): Any amino acid
  MOD_RES (1704)..(1704): Any amino acid
  MOD_RES (1709)..(1709): Any amino acid
  MOD_RES (1714)..(1714): Any amino acid
  MOD_RES (1719)..(1719): Any amino acid
  MOD_RES (1724)..(1724): Any amino acid
  MOD_RES (1729)..(1729): Any amino acid
  MOD_RES (1734)..(1734): Any amino acid
  MOD_RES (1739)..(1739): Any amino acid
  MOD_RES (1744)..(1744): Any amino acid
  MOD_RES (1749)..(1749): Any amino acid
  MOD_RES (1754)..(1754): Any amino acid
  MOD_RES (1759)..(1759): Any amino acid
  MOD_RES (1764)..(1764): Any amino acid
  MOD_RES (1769)..(1769): Any amino acid
  MOD_RES (1774)..(1774): Any amino acid
  MOD_RES (1779)..(1779): Any amino acid
  MOD_RES (1784)..(1784): Any amino acid
  MOD_RES (1789)..(1789): Any amino acid
  MOD_RES (1794)..(1794): Any amino acid
  MOD_RES (1799)..(1799): Any amino acid
  MOD_RES (1804)..(1804): Any amino acid
  MOD_RES (1809)..(1809): Any amino acid
  MOD_RES (1814)..(1814): Any amino acid
  MOD_RES (1819)..(1819): Any amino acid
  MOD_RES (1824)..(1824): Any amino acid
  MOD_RES (1829)..(1829): Any amino acid
  MOD_RES (1834)..(1834): Any amino acid
  MOD_RES (1839)..(1839): Any amino acid
  MOD_RES (1844)..(1844): Any amino acid
  MOD_RES (1849)..(1849): Any amino acid
  MOD_RES (1854)..(1854): Any amino acid
  MOD_RES (1859)..(1859): Any amino acid
  MOD_RES (1864)..(1864): Any amino acid
  MOD_RES (1869)..(1869): Any amino acid
  MOD_RES (1874)..(1874): Any amino acid
  MOD_RES (1879)..(1879): Any amino acid
  MOD_RES (1884)..(1884): Any amino acid
  MOD_RES (1889)..(1889): Any amino acid
  MOD_RES (1894)..(1894): Any amino acid
  MOD_RES (1899)..(1899): Any amino acid
  MOD_RES (1904)..(1904): Any amino acid
  MOD_RES (1909)..(1909): Any amino acid
  MOD_RES (1914)..(1914): Any amino acid
  MOD_RES (1919)..(1919): Any amino acid
  MOD_RES (1924)..(1924): Any amino acid
  MOD_RES (1929)..(1929): Any amino acid
  MOD_RES (1934)..(1934): Any amino acid
  MOD_RES (1939)..(1939): Any amino acid
  MOD_RES (1944)..(1944): Any amino acid
  MOD_RES (1949)..(1949): Any amino acid
  MOD_RES (1954)..(1954): Any amino acid
  MOD_RES (1959)..(1959): Any amino acid
  MOD_RES (1964)..(1964): Any amino acid
  MOD_RES (1969)..(1969): Any amino acid
  MOD_RES (1974)..(1974): Any amino acid
  MOD_RES (1979)..(1979): Any amino acid
  MOD_RES (1984)..(1984): Any amino acid
  MOD_RES (1989)..(1989): Any amino acid
  MOD_RES (1994)..(1994): Any amino acid
  MOD_RES (1999)..(1999): Any amino acid
  See specification as filed for detailed description of substitutions and preferred embodiments
VPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXG

SEQ ID NO: 9 - protein, 1113 aa, Artificial Sequence
  Description of Artificial Sequence Synthetic polypeptide
MVLSPADKTNVKAAWGKVGAHAGEYGAEALERMFLSFPTTKTYFPHFDLSHGSAQVKGHGKKVADALTNAVAHVDDMPNALSALSDLHAHKLRVDPVNFKLLSHCLLVTLAAHLPAEFTPAVHASLDKFLASVSTVLTSKYRGLVPRGSGHGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGY

SEQ ID NO: 10 - protein, 1118 aa, Artificial Sequence
  Description of Artificial Sequence Synthetic polypeptide
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYHGLVPRGSGHGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGY

SEQ ID NO: 11 - protein, 1113 aa, Artificial Sequence
  Description of Artificial Sequence Synthetic polypeptide
MVLSPADKTNVKAAWGKVGAHAGEYGAEALERMFLSFPTTKTYFPHFDLSHGSAQVKGHGKKVADALTNAVAHVDDMPNALSALSDLHAHKLRVDPVNFKLLSHCLLVTLAAHLPAEFTPAVHASLDKFLASVSTVLTSKYRGLVPRGSGHGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGY

SEQ ID NO: 12 - protein, 1118 aa, Artificial Sequence
  Description of Artificial Sequence Synthetic polypeptide
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYHGLVPRGSGHGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGY

SEQ ID NO: 13 - protein, 633 aa, Artificial Sequence
  Description of Artificial Sequence Synthetic polypeptide
MVLSPADKTNVKAAWGKVGAHAGEYGAEALERMFLSFPTTKTYFPHFDLSHGSAQVKGHGKKVADALTNAVAHVDDMPNALSALSDLHAHKLRVDPVNFKLLSHCLLVTLAAHLPAEFTPAVHASLDKFLASVSTVLTSKYRGLVPRGSGHGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGY

SEQ ID NO: 14 - protein, 638 aa, Artificial Sequence
  Description of Artificial Sequence Synthetic polypeptide
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYHGLVPRGSGHGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGVPGAGY

SEQ ID NO: 15 - protein, 142 aa, Canis lupus familiaris
MVLSPADKTNIKSTWDKIGGHAGDYGGEALDRTFQSFPTTKTYFPHFDLSPGSAQVKAHGKKVADALTTAVAHLDDLPGALSALSDLHAYKLRVDPVNFKLLSHCLLVTLACHHPTEFTPAVHASLDKFFAAVSTVLTSKYR

SEQ ID NO: 16 - protein, 147 aa, Canis lupus familiaris
MVHLTAEEKSLVSGLWGKVNVDEVGGEALGRLLIVYPWTQRFFDSFGDLSTPDAVMSNAKVKAHGKKVLNSFSDGLKNLDNLKGTFAKLSELHCDKLHVDPENFKLLGNVLVCVLAHHFGKEFTPQVQAAYQKVVAGVANALAHKYH

SEQ ID NO: 17 - protein, 142 aa, Felis catus
MVLSAADKSNVKACWGKIGSHAGEYGAEALERTFCSFPTTKTYFPHFDLSHGSAQVKAHGQKVADALTQAVAHMDDLPTAMSALSDLHAYKLRVDPVNFKFLSHCLLVTLACHHPAEFTPAVHASLDKFFSAVSTVLTSKYR

SEQ ID NO: 18 - protein, 147 aa, Felis catus
MGFLSAEEKGMVNGLWGKVNVDEVGGEALGRLLVVYPWTQRFFQSFGDLSSADAIMSNSKVKAHGKKVLNSFSDGLKNIDDLKGAFAKLSELHCDKLHVDPENFRLLGNVLVCVLAHHFGHDFNPQVQAAFQKVVAGVANALAHKYH

SEQ ID NO: 19 - protein, 142 aa, Equus caballus
MVLSAADKTNVKAAWSKVGGHAGEFGAEALERMFLGFPTTKTYFPHFDLSHGSAQVKAHGKKVGDALTLAVGHLDDLPGALSNLSDLHAHKLRVDPVNFKLLSHCLLSTLAVHLPNDFTPAVHASLDKFLSSVSTVLTSKYR

SEQ ID NO: 20 - protein, 147 aa, Equus caballus
MVQLSGEEKAAVLALWDKVNEEEVGGEALGRLLVVYPWTQRFFDSFGDLSNPGAVMGNPKVKAHGKKVLHSFGEGVHHLDNLKGTFAALSELHCDKLHVDPENFRLLGNVLVVVLARHFGKDFTPELQASYQKVVAGVANALAHKYH